(19) United States
(12) Patent Application Publication
(10) Pub. No.: US 2008/0058274 A1
(43) Pub. Date: Mar. 6, 2008

(54) Combination Therapy

(76) Inventors: Yechezkel Barenholz, Jerusalem (IL); Elena Khazanov, Beit-Shemesh (IL)

Correspondence Address: NATH & ASSOCIATES, 112 South West Street, Alexandria, VA, 22314, US

(21) Appl. No.: 11/667,671

(22) PCT Filed: Nov. 15, 2005
(86) PCT No.: PCT/IL05/01200

§ 371 Date: Oct. 2, 2007

Related U.S. Application Data
(60) Provisional application No. 60/627,281, filed on Nov. 15, 2004.

(51) Int. Cl. A61K 31/13; A61K 31/70; A61P 43/00

(52) U.S. Cl. 514/34; 514/579

(57) ABSTRACT

The present invention concerns a new medical treatment involving the combination of two active entities, as well as pharmaceutical compositions comprising the two active entities. Specifically, the invention provides a pharmaceutical composition comprising a stable lipid assembly comprising as a first active entity an apoptosis-affecting lipid which does not self-aggregate in a polar environment to form liposomes and a lipopolymer. The pharmaceutical composition further comprises, as the second active entity, a cytotoxic amphipathic weak base drug carried by the lipid assembly or by a different liposome. According to one embodiment, the apoptotic-affecting lipid is a pro-apoptotic lipid. A preferred pro-apoptotic lipid is ceramide, preferably C6-ceramide. The cytotoxic amphipathic weak base drug is preferably doxorubicin or a biologically active, anthracyline-based doxorubicin analog thereof.

### FIELD OF THE INVENTION

This invention relates to combined therapy, and in particular to treatment of proliferative disorders by combination of two or more therapeutic agents.

### LIST OF PRIOR ART

The following is a list of art which is considered to be pertinent for describing the state of the art in the field of the invention.

Barenholz et al. WO 2004/087097

Vento, R. M. et al. Mol. Cell. Biochem. 185:7-153 (1998);

Ogretmen, B. D. et al J. Biol. Chem., 276:24901-24910 (2001);

Hannun Y. A. et al. Biochimica et Biophysica Acta 1585:114-125 (2002);

Ogretmen, B. D. et al. J. Biol. Chem. 276:24901-24910 (2001);

Mueller, H. and Eppenberger, U. Anticancer Res. 16:3845-3848 (1996);

Senchenkov, A. et al. J. Natl. Cancer Inst. 93:347-357 (2001);

Z. Cai, Z. et al. J. Biol. Chem. 272:6918-6926 (1997);

Charles A G. et. al., Cancer Chemother Pharmacol 47(5):444-450 (2001);

Mehta S. et al. Cancer Chemother Pharmacol 46(2):85-92 (2000);

Lucci A. et al. Int J Oncol 15(3):541-546 (1999);

Cabot M C. et al. FEBS Lett 394(2):129-131 (1996);

Lavie T. et al. J Biol Chem 272(3):1682-1687 (1997);

Lucci A. et al. Cancer 86(2):300-311 (1999);

Lucci A. Int J Oncol 15(3):541-546 (1997);

Lofgren and Pasher, Chem. Phys. Lipids, 20(4):273-284, (1977);

Carrer and Maggio, Biochim. Biophys Acta, 1514(1):87-99, (2001).

### BACKGROUND OF THE INVENTION

Many lipids are bioactive, i.e. are directly or indirectly involved in signal transduction pathways that mediate cell growth, differentiation, cell death and many other cell functions, as exemplified by diacylglycerols (DAG), ceramides (Cer), sphingosine (Sph), sphingosine-1-phosphate (SIP), ceramide-1-phosphate (C-1-P), di- and trimethylsphingosine (DMS and TMS, respectively). Most of these lipids or their derivatives have the potential to have a therapeutic effect either as standalone drugs or as a support to other drugs.

The discovery of pro-apoptotic properties of ceramides [Vento, R. M. et al. Mol. Cell. Biochem. 185:7-153 (1998)] and the finding that ceramides inactivate telomerase activity and, therefore, might be cancer-specific [Ogretmen, B. D. et al J. Biol. Chem., 276:24901-24910 (2001)] made them an attractive candidates for antitumor therapy alone, as well as in combination with chemotherapeutic agents, in an attempt to overcome some of obstacles of chemotherapy. The role of ceramide in apoptosis is discussed in numerous publications. Hannun Y. A et al. summarizes insights from studies of Cer metabolism, topology and effector action, identification of several genes for enzymes of ceramide metabolism, ceramide analysis etc. [Hannun Y. A. et al. Biochimica et Biophysica Acta 1585:114-125 (2002)].

The demonstration of a role of ceramide in anti-proliferative processes [Ogretmen, B. D. et al. J. Biol. Chem. 276:24901-24910 (2001)] implies that a defect in ceramide generation or in ceramide effector mechanisms could be involved in conferring a survival advantage to cancer cells. Other studies [Mueller, H. and Eppenberger, U. Anticancer Res. 16:3845-3848 (1996)] suggest that dysfunctional metabolism of ceramide which contributes to reduction in the level of ceramide is implicated in multi-drug (MD) resistance. A number of clinically important cytotoxic agents appear to be effective because of their ability to activate ceramide-activated pathways in cancer cells by activating ceramide synthase or sphingomyelinase enzymes, or by inhibition of glucosylceramide synthase (GCS) activity. It was shown that TNF-α-resistant MCF-7 breast cancer cells have been characterized by inability of their sphingomyelinases to generate ceramide [Senchenkov, A. et al. J. Natl. Cancer Inst. 93:347-357 (2001)]. Also, the human ovarian adenocarcinoma cell line NIH:OVCAR-3, established from a patient resistant to doxorubicin (DXN), mephalan, and cisplatin, expresses high levels of glucosylceramide, which agrees with high levels of GCS [Z. Cai, Z. et al. J. Biol. Chem. 272:6918-6926 (1997)].

Further, many clinically important cytotoxic agents have suggested to be effective by synergizing with ceramide-mediated apoptotic signaling pathway in cancer cells. The cytotoxic effect of taxol was linked to the de novo synthesis of ceramide in MDA-MB 468 human breast cancer cells, and taxol-dependent cytotoxicity was abolished when ceramide formation was blocked using L-cycloserine, an inhibitor of de novo ceramide synthesis. Moreover, exogenous ceramide synergistically augmented taxol-induction of apoptosis. [Charles A G. et. al., Cancer Chemother Pharmacol 47(5):444-450 (2001); Mehta S. et al. Cancer Chemother Pharmacol 46(2):85-92 (2000). Doxorubicin was also shown to promote ceramide formation and apoptosis in breast cancer cells [Lucci A. et al. Int J Oncol 15(3):541-546 (1999)]. Tamoxifen was shown to increase cellular ceramide levels by blocking conversion of ceramide to glucosylceramide, which was independent of estrogen receptor status [Cabot M C. et al. FEBS Lett 394(2):129-131 (1996); Lavie T. et al. J Biol Chem 272(3):1682-1687 (1997)]. Furthermore, the combination of tamoxifen with agents, such as doxorubicin or cyclosporine A analogue, was shown to exert synergistic effects on ceramide formation [Lucci A. et al. Cancer 86(2):300-311 (1999)].

Multi-drug resistant (MDR) cancers was also suggested to be linked to augmented ceramide metabolism. Exposure to doxorubicin increases ceramide levels in drug-sensitive MCF-7 breast cancer cells, but not in the doxorubicin-resistant MCF-7-AdrR cells [Lucci A. Int J Oncol 15(3):541-546 (1997)]. Additionally, it was shown that while neither C₆-Cer nor tamoxifen (a known inhibitor of GlcCer synthase) was cytotoxic alone, the addition of tamoxifen to the C₆-Cer treatment regimen decreased MCF-7-AdrR cell viability and elicited apoptosis. Further treatment of these cells with Adriamycin stimulated an increase in endogenous ceramide levels only if co-administered with tamoxifen, in which case augmented ceramide levels correlated with a further decline in cell viability. However, as described, since MCF-7-AdrR cells have a high level of GlcCer synthase activity, these cells are suspected to display resistance to exogenous cell-permeable ceramide as well as chemotherapeutic agents (i.e., doxorubicin and adriamycin) through metabolism of ceramide into GlcCer.

Thus, it was suggested that elevating intracellular ceramide levels, either by exogenous administration alone or in combination with chemotherapeutic agents, or by targeting ceramide metabolism and cell death signaling pathways, is an attractive clinical treatment strategy for therapy of sensitive tumors as well as for overcoming drug resistance.

However, with most of these bioactive lipids, an obstacle to such application in vivo is the lack of ability to administer and/or to deliver these molecules in a way that will retain their bioactivity. Most of these bioactive lipids are not soluble in aqueous phase; some such as DAG and ceramides, are difficult to disperse in a stable form in relevant media; some when dispersed as micelles (S1P, Sph) disintegrate in biological fluids such as blood; most of them when incorporated into liposomes cause the liposome to be physically unstable.

A recent development involves the incorporation of such bioactive lipids in vesicles' membrane to facilitate their delivery into cells. WO2004/087097 describes an organized collection of lipids forming lipid assemblies, comprising a specific combination of a bioactive lipid (which cannot self-assemble to form stable vesicles), a lipopolymer, and a lipid matrix (acting as a backbone for the stable assembly). The lipid assemblies were found to be chemically and physically stable under storage condition of 4° C., for at least 6 months, and in biological fluids. A specific group of bioactive lipids that cannot self-assemble to form stable vesicles and is specifically discussed in WO 2004/08797 includes the ceramides. Ceramides are lipids composed of fatty acids linked by an amide bond to the amino group of a long chain sphingoid base and are known to be key intermediates in the biosynthesis of sphingolipids [Lofgren and Pasher, Chem. Phys. Lipids, 20(4):273-284, (1977); Carrer and Maggio, Biochim. Biophys Acta, 1514(1):87-99, (2001)].

DOXIL® is doxorubicin HCl encapsulated in long-circulating STEALTH® liposomes which is approved for the treatment of ovarian cancer. The STEALTH® liposomes of DOXIL® are formulated with surface-bound methoxypolyethylene glycol (MPEG), a process often referred to as pegylation, to protect liposomes from detection by the mononuclear phagocyte system (MPS) and to increase blood circulation time. The mechanism of action of doxorubicin HCl is thought to be related to its ability to bind DNA and inhibit nucleic acid synthesis. Studies have demonstrated rapid cell penetration of DXN and perinuclear chromatin binding followed by rapid inhibition of mitotic activity and nucleic acid synthesis, and induction of mutagenesis and chromosomal aberrations.

STEALTH® liposomes have a half-life of approximately 55 hours in humans. They are stable in blood, and direct measurement of liposomal doxorubicin shows that at least 90% of the drug (the assay used cannot quantify less than 5-10% free doxorubicin) remains liposome-encapsulated during circulation.

It is hypothesized that because of their small size (<100 nm) and persistence in the circulation, the pegylated DOXIL® liposomes are able to extravasate from the altered and often compromised vasculature of tumors and penetrate the tumor itself. This hypothesis is supported by studies using colloidal gold-containing STEALTH® liposomes, which can be visualized microscopically.

### SUMMARY OF THE INVENTION

The present invention is based on the finding that treating cancer cells with a combination of liposomes carrying a cytotoxic drug (Doxil®) and liposomes, carrying in their lipid membrane a pro-apoptotic lipid (ceramide), produced a beneficial additive effect, i.e. inhibition of proliferation of the cells which was at least sum of effects obtained when treating the same cells with the liposomal cytotoxic drug alone or the liposomal pro-apoptotic lipid alone.

The present invention is further based on the finding that a composition comprising liposomes having the pro-apoptotic lipid (ceramide) in their membrane, and a cytotoxic drug (doxorubicin) in the intraliposomal aqueous phasewere able, when administered to tumor-bearing animals, to achieve 100% survival rate for the entire tested period.

Thus, according to a first of its aspects, the present invention provides a pharmaceutical composition comprising:

(a) a stable lipid assembly comprising:
i) an apoptosis affecting lipid, which does not self-aggregates in a polar environment to form liposomes;
ii) a lipopolymer;

(b) a liposome carrying a cytotoxic, amphipathic weak base drug.

The Lipid assembly as used herein denotes an organized collection of lipids forming inter alia, micelles or liposomes, preferably this term denotes liposomes.

The term “apoptosis-affecting lipid” denotes a lipid which has an effect of either inducing apoptosis (pro-apoptotic lipid) or inhibiting apoptosis (anti-apoptotic lipid). The apoptotic activity of the apoptosis-affecting lipids according to the invention refers to any measurable apoptosis, as indicated by well known parameters, such as, exposure of phosphatidylserine at the external surface of the cell's plasma membrane, rounding the cells, chromatic degradation and condensation, breaking of the nucleus, plasma membrane breaking into apoptotic bodies) as exhibited on a biological target site. The biological target site according the invention may include a cell, tissue or organ or a component thereof (e.g. intracellular component).According to one embodiment of the invention, referred to herein by the term the “mixed population embodiment” the lipid assemblies (preferably liposomes) comprising the apoptosis affecting lipid(s), and the liposomes carrying the cytotoxic-drug are two populations which are mixed to give the pharmaceutical composition of the invention. The ratio between the two populations will depend inter alia, on the type of the drug, its therapeutic dose and treatment regiment. Those versed in the art of pharmacy will know how to determine this ratio based on the type of drug.

According to yet another embodiment of the invention, referred to herein by the term the “single population embodiment”, the lipid assemblies comprise the apoptosis-affecting lipid in their lipid-based membranes and these lipid assemblies are the same lipid structure that carries the cytotoxic drug, such that a single type of lipid-based population is used.

Thus, the present invention also provides a pharmaceutical composition comprising a stable lipid assembly comprising an apoptosis-affecting, which does not self-aggregates in a polar environment, to form liposomes and a lipopolymer; and said lipid assembly carries a cytotoxic, amphipathic weak base drug.

Preferably the lipid assembly is a liposome.

According to a preferred embodiment, the apoptosis-affecting lipid is a pro-apoptotic lipid and the pharmaceutical compositions of the invention are preferably for the treatment of proliferative or hyperproliferative conditions.

The invention also provides a method for treating a subject having a proliferative or hyperproliferative condition. In accordance with the “mixed population” embodiment of the invention, there is provided a method of treating a subject having a proliferative or hyperproliferative condition comprising administering to the subject a pharmaceutical composition pharmaceutical composition comprising:

(a) a stable lipid assembly comprising:
i) an apoptosis affecting lipid which does not self-aggregates in a polar environment, to form liposomes;
ii) a lipopolymer;

(b) a liposome carrying a cytotoxic, amphipathic weak base drug.

In accordance with the “single population” embodiment of the invention, there is provided a method for treating a subject having a proliferative or hyperproliferative condition, the method comprises administering to the subject a pharmaceutical composition comprising a stable lipid assembly comprising an apoptosis-affecting lipid which does not self-aggregates, in a polar environment, to form liposomes; a lipopolymer; and a cytotoxic, amphipathic weak base drug carried by the lipid assembly.

In accordance with the invention a further method is provided for treating a subject having a proliferative or hyperproliferative condition and being treated with liposomes comprising doxorubicin or a biologically active, anthracycline-based doxorubicin analog, the method comprises administering to said subject an effective amount of liposomes comprising in their membrane a lipopolymer and ceramide.

Further in accordance with the invention, a method is provided for treating a subject having a proliferative or hyperproliferative condition and being treated with liposomes comprising in their membrane a lipopolymer and ceramide, the method comprises administering to the subject liposomes comprising doxorubicin or a biologically active, anthracycline-based doxorubicin analog.

Preferably the administration should be co-administration (which means either simultaneous administration or administration within a very short interval). If the two populations are administered separately, it is preferable that the time interval between administrations is not more than several hours and preferably, the lipid-assembly carrying the apoptosis affecting lipid is administered to the subject before administration of the cytotoxic drug.

In accordance with the “mixed population” embodiment of the invention, there is also provided the use of a stable lipid assembly comprising an apoptosis-affecting lipid, which does not self-aggregates in a polar environment to form liposomes and a lipopolymer together with liposomes carrying a cytotoxic, amphipathic weak base drug, for the preparation of a pharmaceutical composition.

There is further provided, in accordance with the “single population” embodiment of the invention, the use of a stable lipid assembly comprising in its lipid membrane an apoptosis-affecting lipid which does not self-aggregates in a polar environment to form a liposome, together with a cytotoxic, amphipathic weak base drug to be carried by the lipid assembly, for the preparation of a pharmaceutical composition.

Preferably the pharmaceutical compositions are for the treatment of a proliferative or hyperproliferative condition.

According to a preferred embodiment, the apoptosis-affecting lipid is a pro-apoptotic lipid, more preferably ceramide, specifically, C₆ ceramide and the cytotoxic drug is an anthracycline-based compound, preferably doxorubicin or an anthracycline-based analog thereof as defined below.

Thus, according to the “mixed population” embodiment of the invention, one specific composition to be employed is that comprising a mixture of first population of liposomes having a lipid membrane comprising a lipid matrix, C₆ ceramide and a lipopolymer, and a second population of liposomes encapsulating in the intraliposomal aqueous phase doxorubicin or a biologically active anthracycline-based analog of doxorubicin.

In accordance with the “single population” embodiment of the invention one specific composition to be employed is that comprising one species of liposomes being a liposome having a lipid membrane comprising a lipid matrix, C₆ ceramide and a lipopolymer, the liposome encapsulating in the intraliposomal aqueous phase doxorubicin or a biologically active anthracycline-based analog of doxorubicin.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a bar graph showing the cytotoxic activity of liposomal C₆Cer (white bar), doxorubicin (DXN) (grey bar) or their combination (black bar) against DXN-resistant M109R tumor cell line. Tumor cells were incubated with the different formulations for 96 hr and percent survival was measured with the aid of MB assay.

FIG. 2 is a graph showing the therapeutic activity of sterically stabilized C₆Cer-DXN(C₆Cer-DXN-SSL) as compared to Doxil. Percent (%) survival of BALB/c mice inoculated i.p. with 1*10⁶ C-26 colon carcinomas and treated as described is exhibited.

FIG. 3 is a graph showing the therapeutic activity of C₆Cer-DXN-SSL compared to Doxil or free DXN. Percent (%) survival of BALB/c mice inoculated i.p. with 1*10⁶ C-26 colon carcinomas and treated as described is exhibited.

FIGS. 4A-4B are graphs exhibiting the pharmacokinetics (FIG. 4A) and bio-distribution (FIG. 4B) of C₆Cer-DXN-SSL (gray bar) as compared to Doxil (white bar) or free DXN (black bar).

### DETAILED DESCRIPTION OF THE INVENTION

The present invention concerns the development of a novel combination therapy leading to a therapeutic effect superior to the effect obtained when applying each individual therapy alone. It was shown that combination therapy resulted in the non-expected, outstanding highest possible therapeutic effect (100% survival).

As shown by the non-limiting examples provided herein, when formulating together liposomes which include a significant level (>5 mole %) of a bioactive lipid (the apoptosis-affecting lipid, specifically pro-apoptotic) embedded in the liposome's membrane and a cytotoxic drug, such as the anti-cancer drug doxorubicin, in the intraliposome aqueous phase of a liposome (either the same or different liposome), a stable liposomal composition is obtained which when tested, in vitro as well as in vivo, exhibited a beneficial therapeutic effect.

Thus, the present invention provides a pharmaceutical composition comprising a lipid assembly, preferably liposomes, comprising an apoptotsis-affecting lipid which aggregates, in a polar environment, to a state other than liposomes, (i.e. a lipid which does not spontaneously self-aggregate in a polar, aqueous, environment into liposomes); a lipopolymer and a cytotoxic, amphipathic weak base drug. As indicated above, the cytotoxic drug may be in a different population of lipid assemblies (according to the “mixed population” embodiment) or in the same population of lipid assemblies as the apoptosis-affecting lipid (according to the “single population” embodiment).

The lipid assembly carrying the apoptosis-affecting lipid is preferably a stable lipid assembly.

The term “stable lipid assembly” as used herein denotes an assembly being chemically and physically stable under storage conditions (4° C., for at least 6 months) and also stable in biological fluids. This term also encompass assemblies, which in the presence of a lipopolymer, the apoptosis-affecting lipid (which by itself does not form liposomes), so that during storage the integrity and composition of the lipid assembly is substantially unaltered. The stability of the assembly is accomplished by the combination of a apoptosis-affecting lipid with the lipopolymer, i.e. in the absence of the lipopolymer, a substantial portion of the apoptosis-affecting lipid initially loaded into the assembly (i.e. upon formation of the assembly) is removed therefrom within a short time after storage and/or aggregation of lipids occurs. As a result, the assembly is toxic and/or the injection dose does not carry sufficient (desired) amount of the apoptosis-affecting lipid to the target site and the assembly is not effective to achieve the desired biological/therapeutic effect.

The apoptosis-affecting lipid may be any naturally occurring, synthetic and semi-synthetic amphiphile having a hydrophobic region, comprising, one or more, long hydrocarbon chains and a polar, ionic or non-ionic headgroup, wherein the atomic mass ratio between the headgroup and hydrophobic region is less than 0.3. Such amphiphiles may also be defined by their geometrical structure, typically being in the shape of a truncated inverted cone. Alternatively, or in addition, non-liposome forming lipids may be defined by their packing parameter, being greater than 1.

Further, the apoptosis-affecting lipids according to the invention are such that when mixed alone in a polar (aqueous) environment, tend to aggregate to a state other than liposomes, i.e. when on their own (not mixed with other lipids) do not form liposomes. These non-liposomal states include, for example, micelles, inverted hexagonal phases or assemblies of a wide range of sizes or long and thin tubular structures or undefined precipitates. The apoptosis-affecting lipids are typically embedded with their hydrocarbon chains in parallel to the hydrocarbon chains of other components (such as phospholipids) of the assembly.

Non-limiting examples of pro-apoptotic lipid include ceramides, ceramines, sphinganines, sphinganine-1-phosphate, di- or tri-alkylshpingosines and their structural biologically functional analogs.

A preferred group of bioactive lipids may be defined by the following general chemical formula (I):

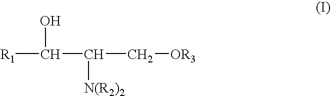

wherein
R ₁ represent a C₂-C₂₆, saturated or unsaturated, branched or unbranched, aliphatic chain, the aliphatic chain may be substituted with one or more hydroxyl or cycloalkyl groups and may consist of a cycloalkylene moiety;
R₂ which may be the same or different, represents a hydrogen, a C₁-C₂₆ saturated or unsaturated, branched or unbranched chain selected from aliphatic, aliphatic carbonyl; a cycloalcylene-containing aliphatic chain, the aliphatic chain may be substituted with an aryl, arylalkyl or arylalkenyl group;
R₃ represents a hydrogen, a methyl, ethyl, ethenyl, a carbohydrate or a phosphate group.

A specific group of pro-apoptotic lipids is that in which R₁ is a C₁₅ aliphatic chain, a first R₂ is hydrogen, a second R₂ is as defined above, and R₃ is hydrogen.

A more specific group of the apoptosis-affecting lipids are those in which R₁ is a C₁₅ unsaturated aliphatic chain, the un-saturation, i.e. double bond, being between carbon atoms C₁-C₂ of R₁ (which corresponds to positions C₄-C₅ of a sphingoid base), a first R₂ is hydrogen, a second R₂ group is C₁-C₂₆ saturated or non-saturated, optionally hydroxyl substituted (once or more) aliphatic chain, and R₃ is hydrogen.

A preferred group of apoptosis-affecting lipids are the ceramides. Ceramides of preferred choice are the short chain (C₂-C₈) ceramide analogs and preferably C₆ ceramide (C₆Cer).

As known to those versed in the art, there are difficulties in the in vivo delivery of the various ceramides, including short chain cell permeable analogs of ceramides (e.g. C₂-, C₆-, C₄- or C₈-ceramide). The effectiveness of delivery is limited by the molecules hydrophobicity which leads to the formation of large aggregates upon in vivo delivery. [Radin N S Eur J Biochem 268:193-204 (2001)]. Thus, a critical need for improved delivery systems to maximize intracellular ceramide accumulation upon systemic administration was identified.

The therapeutically effective incorporation of ceramides (long and short chain) in lipid membranes of liposomes was recently accomplished by the inventors (WO2004/087097). The resulting ceramide-bearing liposomes were stable upon storage (see definition of stability above) and effective in inducing apoptosis. Steric stabilization of the lipid assemblies was achieved, inter alia, by the incorporation of a lipopolymer in the lipid membrane.

As defined hereinabove, the lipid assemblies further comprise a lipopolymer. The term “lipopolymer” as used herein denotes a lipid substance modified at its polar headgroup with a hydrophilic polymer. The lipopolymer according to the invention may be further defined by the atomic mass ratio between the polymer headgroup and the lipid hydrophobic region, being at least 1.5. Preferably, the lipopolymers of the invention are such that the level of water tightly bound to the headgroup is about 60 molecules of water per lipopolymer molecule. The level of water tightly bound to the headgroup is determined as described in Tirosh O. et. al [Tirosh O. et. al Biophysical Journal, 74, 1371-1379 (1998)]. In general, Tirosh et al. show that the calculation of the accessible surface area of a lipopolymer, such as a PEG molecule, from the specific volume data for the PEG and its components is at least three water molecules per PEG repeated unit. Thus, a whole ⁷⁵⁰PEG molecule, having a degree of polymerization of 15, binds ˜60 water molecules and ^{2k}PEG molecule, having a degree of polymerization of 46, binds ˜142 water molecules.

The polymer headgroup of the lipopolymer is typically water-soluble and may be covalently or non-covalently attached to a hydrophobic lipid region. The lipopolymers which may be employed in the context of the present invention are well known in the art and are tolerated in vivo without toxic effects (i.e. are biocompatible). Lipopolymers such as those employed by the present invention are known to be effective for forming long-circulating liposomes.

Lipopolymers according to the invention comprise preferably lipids, typically, modified at their head to include a polymer having a molecular weight equal or above 750 Da. The headgroup may be polar or apolar, however, is preferably a polar head group to which a large (>750 Da) highly hydrated (at least 60 molecules of water per headgroup) flexible polymer is attached. The attachment of the hydrophilic polymer headgroup to the lipid region may be a covalent or non-covalent attachment, however, is preferably via the formation of a covalent bond (optionally via a linker).

The outermost surface coating of hydrophilic polymer chains is effective to provide a liposome with a long blood circulation lifetime in vivo. The lipopolymer may be introduced into the liposome by two different ways: (a) either by adding the lipopolymer to a lipid mixture forming the liposome. The lipopolymer will be incorporated and exposed at the inner and outer leaflets of the liposome bilayer [Uster P. S. et al. FEBBS Letters 386:243 (1996)]; (b) or by firstly prepare the liposome and then incorporate the lipopolymers to the external leaflet of the pre-formed liposome either by incubation at temperature above the Tm of the lipopolymer and liposome-forming lipids, or by short term exposure to microwave irradiation.

Preparation of vesicles Composed of liposome-forming lipids and derivatization of such lipids with hydrophilic polymers (thereby forming lipopolymers) has been described, for example by Tirosh et al. [Tirosh et al., Biopys. J., 74(3):1371-1379, (1998)] and in U.S. Pat. Nos. 5,013,556; 5,395,619; 5,817,856; 6,043,094, 6,165,501, incorporated herein by reference and in WO 98/07409. The lipopolymers may be non-ionic lipopolymers (also referred to at times as neutral lipopolymers or uncharged lipopolymers) or lipopolymers having a net negative or a net positive charge.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers.

While the lipids derivatized into lipopolymers may be neutral, negatively charged, as well as positively charged, i.e. there is no restriction to a specific (or no) charge, the most commonly used and commercially available lipids derivatized into lipopolymers are those based on phosphatidyl ethanolamine (PE), usually, distearylphosphatidylethanolamine (DSPE).

A specific family of lipopolymers employed by the invention include monomethylated PEG attached to DSPE (with different lengths of PEG chains, the methylated PEG referred to herein by the abbreviation PEG) in which the PEG polymer is linked to the lipid via a carbamate linkage resulting in a negatively charged lipopolymer. Other lipopolymers are the neutral methyl polyethyleneglycol distearoylglycerol (mPEG-DSG) and the neutral methyl polyethyleneglycol oxycarbonyl-3-amino-1,2-propanediol distearoylester (mPEG-DS) [Garbuzenko O. et al., Langmuir. 21:2560-2568 (2005)]. Another lipopolymer is the phosphatidic acid PEG (PA-PEG). The PEG moiety preferably has a molecular weight of the head group is from about 750 Da to about 20,000 Da. More preferably, the molecular weight is from about 750 Da to about 12,000 Da and most preferably between about 1,000 Da to about 5,000 Da. One specific PEG-DSPE employed herein is that wherein PEG has a molecular weight of 2000 Da, designated herein ²⁰⁰⁰PEG-DSPE or ^{2k}PEG-DSPE.

In addition to the contribution of the lipopolymer to the stabilization of the lipid assembly comprising the apoptosis-affecting lipid, the lipopolymer provide a surface coating of hydrophilic polymer chains on both the inner and outer surfaces of the liposome lipid bilayer membranes. The outermost surface coating of hydrophilic polymer chains is effective to provide the lipid assembly with a long blood circulation lifetime in vivo. In case of liposome formation, the inner coating of hydrophilic polymer chains may extend into the aqueous compartments in the liposomes, between the lipid lamella and into the central core compartment, which may contain additional therapeutic agents.

In addition to the apoptosis-affecting lipid and lipopolymer, the lipid assembly comprises other components, such as other lipids, all together forming a lipid matrix (a scaffold). It is to be understood that the lipid matrix may comprise a single lipid (in addition to the apoptosis-affecting lipid) or a combination of lipids forming the lipid lamella (e.g. the liposomes bilayer). When forming a liposome, the combination of lipids forming the lipid matrix may be defined by their additive packing parameter being in the range of 0.74 and 1.0. By way of comparison, the packing parameter of the bioactive lipid is greater than 1.0.

The term “additive effective packing parameter” refers to the relative (mole % weighted) contribution of the packing parameter of each constituent of the liposome to the average (i.e. the weighted sum) packing parameter of the final lipid composition which constitute the liposome. The fact that the additive effective packing parameter of the structure is within the range of 0.74-1.0, in case of liposomes, indicates that preferably a liposome is formed and that the combination of all constituents used to form the liposome's lamella results in the formation of stable liposomes.

The lipids forming the lipid matrix typically include one or two hydrophobic acyl chains, which may be combined with a steroid group, and may contain a chemically reactive group, (such as an amine, acid, ester, aldehyde or alcohol) at the polar head group. One group of lipids forming the matrix includes physiologically acceptable liposome forming lipids. Liposome-forming lipids are typically those having a glycerol backbone wherein at least two of the hydroxyl groups is substituted with acyl chains and a third hydroxyl group is replaced with a phosphate group to which reactive groups may be attached, a combination or derivatives of same and may contain a chemically reactive group as defined above at the headgroup. Typically, the acyl chain(s) is between 14 to about 24 carbon atoms in length, and has varying degrees of saturation being fully, partially or non-hydrogenated lipids. Further, the lipid matrix may be of natural source, semi-synthetic or fully synthetic lipid, and neutral, negatively or positively charged.

According to one embodiment, the liposome forming lipids comprise phospholipids. The phospholipids may be a glycerophospholipid. Examples of glycerophospholipid include, without being limited thereto, three types of lipids: (i) zwiterionic phospholipids, which include, for example, phosphatidylcholine (PC), egg yolk phosphatidylcholine, dimyristoyl phosphatidylcholine (DMPC) sphingomyelin (SM); (ii) negatively charged phospholipids: which include, for example, phosphatidylserine (PS), phosphatidylinositol (PI), phosphatidic acid (PA), phosphatidylglycerol (PG) and dimyristoyl phosphatidylglycerol (DMPG); and (iii) cationic phospholipids, which include, for example, phosphatidylcholine or sphingomyelin of which the phosphomonoester was O-methylated to form the cationic lipids.

A specific phosphatidylcholine employed in accordance with the invention is hydrogenated soybean PC(HSPC).

The lipid assembly may also include other components typically used in the formation of lipid assemblies. For example, cationic lipids may be incorporated in order to produce cationic liposomes. The cationic lipid can be included as a minor component of the lipid assembly or as a major or sole component. Such cationic lipids typically have a lipophilic moiety, such as a sterol, an acyl or diacyl chain, and where the lipid has an overall net positive charge. Preferably, the head group of the lipid carries the positive charge. Monocationic lipids may include, for example, 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy) propyl]-N,N,N-trimethylammonium chloride (DOTMA); 3P[N—(N′,N′-dimethylaminoethane) carbamoly] cholesterol (DC-Chol); and dimethyl-dioctadecylammonium (DDAB).

Examples of polycationic lipids include a similar lipophilic moiety as with the mono cationic lipids, to which polycationic moiety is attached. Exemplary polycationic moieties include spermine or spermidine (as exemplified by DOSPA and DOSPER), or a peptide, such as polylysine or other polyamine lipids. For example, the neutral lipid (DOPE) can be derivatized with polylysine to form a cationic lipid. polycationic lipids include, without being limited thereto, N-[2-[[2,5-bis[3-aminopropyl)amino]-1-oxopentyl]amino]ethyl]-N,N-dimethyl-2,3-bis[(1-oxo-9-octadecenyl)oxy]-1-propanaminium (DOSPA), and ceramide carbamoyl spermine (CCS).

Further, other components suitable for stabilization of the lipid assembly may include, without being limited thereto, sterols and sterol derivatives, such as cholesterol, cholesteryl hemisuccinate, cholesteryl sulfate.

The mole % of each component in the lipid assembly may be determined and selected to achieve a specified degree of fluidity or rigidity, to control the stability of the assembly during storage as well as after delivery, e.g. in serum and to control the rate of release of the pro-apoptotic lipid forming part of the assembly or the cytotoxic drug carried thereby. Lipid assemblies having a more rigid structure, e.g. liposomes in the gel (solid ordered) phase or in a liquid crystalline fluid (liquid disordered) state, are achieved by reducing or eliminating sterols from the lipid composition and by using a relatively rigid lipid, for example, a lipid having a relatively high solid ordered to liquid disordered phase transition temperature, such as, above room temperature. Rigid, i.e., saturated, lipids having long acyl chains, contribute to greater membrane rigidity in the assembly. A good example for such a lipid is HSPC or DSPC. Lipid components, such as cholesterol, are also known to contribute to rigidity in lipid assemblies based on fluid lipids. Such a sterol reduces free volume thereby reducing permeability. Similarly, high lipid fluidity is achieved by incorporation of a relatively fluid lipid, typically one having a relatively low liquid to liquid-crystalline phase transition temperature, for example, at or below room temperature, more preferably, at or below the target body temperature. A good example for such a phospholipid is egg PC.

When the lipid assembly is in the form of a liposome, the liposome may be in the form of multilamellar vesicles (MLV), large unilamellar vesicles (LUV), small unilamellar vesicles (SUV) or multivesicular vesicles (MVV) as well as in other bilayered forms known in the art. The size and lamellarity of the liposome will depend on the manner of preparation and the selection of the type of vesicles to be used will depend on the preferred mode of administration. For systemic therapeutic purposes, preferred injectable liposomes are those in the size range of 50-150 nm in diameter (LUV or SUV [Gabizon A. et al. Cancer Res. 54:987-992 (1994)]); for local treatment larger liposomes, such as MLV or MVV, can also be used [Grant G. et al. Anesthesiology 101:133-137 (2004)].

The pharmaceutical composition of the invention also comprises a cytotoxic, amphipathic weak base drug encapsulated within liposomes (being either a separate population of liposomes than those comprising the apoptosis-affecting lipid, or the same population of liposomes). The amphipathic weak base compound is characterized by its ability to permeate normally nonpermeable membrane under suitable trans-membrane pH and/or ammonium gradient conditions. The loading of amphipathic weak acids and bases was described (see below) General principles of the loading procedures (known by the term “active/remove loading”] concern permeation of the drug via a lipid membrane by the use of a lipid assembly created for loading of amphipathic weak bases with a higher, more acidic pH inside than outside the liposome, such system will naturally try to equilibrate, i.e. to achieve the same pH inside as outside. Whether or not such pH equilibration is possible or how fast it will happen, depends on the chemical properties of the membrane separating the internal aqueous phase from external aqueous phase and on the medium composition. Liposomes, by virtue of their lipid bilayers, present an optimal membrane barrier naturally resisting such equilibration. In by themselves, liposomes may be formed in an appropriate medium such as ammonium ion of which a portion will become, in a sense, encapsulated in liposomes, thus forming the ammonium sulfate containing liposomes having certain inner pH. This pH will depend on the difference between the amount loaded ammonium sulfate inside the liposomes and between the amount of ammonium sulfate outside of liposomes. If the outside and inside amounts are the same, pH in both is identical to the pH of ammonium sulfate solution or to the pH of the buffer/ammonium sulfate if the buffer is added to the ammonium sulfate. If however, the outside ammonium sulfate is substituted, diluted, or exchanged with other salts or with non-electrolite such as dextorse or sucrose, the inside of liposomes react quickly by changing pH toward the acidic side.

Liposomes having an H⁺ and/or ion gradient across the liposome bilayer for use in remote loading can be prepared by a variety of techniques. A typical procedure comprises dissolving a mixture of lipids at a ratio that forms stable liposomes in a suitable organic solvent and evaporated in a vessel to form a thin lipid film. The film is then covered with an aqueous medium containing the solute species that will form the aqueous phase in the liposome interior space. After liposome formation, the vesicles may be sized to achieve a size distribution of liposomes within a selected range, according to known methods.

After sizing, the external medium of the liposomes is treated to produce an ion gradient across the liposome membrane (typically with the same buffer used to form the liposomes), which is typically a higher inside/lower outside ion concentration gradient. This may be done in a variety of ways, e.g., by (i) diluting the external medium, (ii) dialysis against the desired final medium, (iii) gel exclusion chromatography, e.g., using Sephadex G-50, equilibrated in the desired medium which is used for elution, or (iv) repeated high-speed centrifugation and resuspension of pelleted liposomes in the desired final medium. The external medium which is selected will depend on the type of gradient, on the mechanism of gradient formation and the external solute and pH desired, as will now be described.

In the simplest approach for generating an ion and/or H⁺ gradient, the lipids are hydrated and sized in a medium having a selected internal-medium pH. The suspension of the liposomes is titrated until the external liposome mixture reaches the desired final pH, or treated as above to exchange the external phase buffer with one having the desired external pH. For example, the original hydration medium may have a pH of 5.5, in a selected buffer, e.g., glutamate, citrate, succinate, fumarate buffer, and the final external medium may have a pH of 8.5 in the same or different buffer. The common characteristic of these buffers is that they are formed from acids which are essentially liposome impermeable. The internal and external media are preferably selected to contain about the same osmolarity, e.g., by suitable adjustment of the concentration of buffer, salt, or low molecular weight non-electrolyte solute, such as dextrose or sucrose.

In another general approach, the gradient is produced by including in the liposomes, a selected ionophore. To illustrate, liposomes prepared to contain valinomycin in the liposome bilayer are prepared in a potassium buffer, sized, then the external medium exchanged with a sodium buffer, creating a potassium inside/sodium outside gradient. Movement of potassium ions in an inside-to-outside direction in turn generates a lower inside/higher outside pH gradient, presumably due to movement of protons into the liposomes in response to the net electronegative charge across the liposome membranes [Deamer, D. W., et al., Biochim. et Biophys. Acta 274:323 (1972)].

A similar approach is to hydrate the lipid and to size the formed multilamellar liposome in high concentration of magnesium sulfate. The magnesium sulfate gradient is created by dialysis against 20 mM HEPPES buffer, pH 7.4 in sucrose. Then, the A23187 ionophore is added, resulting in outwards transport of the magnesium ion in exchange for two protons for each magnesium ion, plus establishing a inner liposome high/outer liposome low proton gradient [Senske D B et al. (Biochim. Biophys. Acta 1414: 188-204 (1998)].

In another more preferred approach, the proton gradient used for drug loading is produced by creating an ammonium ion gradient across the liposome membrane, as described, for example, in U.S. Pat. Nos. 5,192,549 and 5,316,771, incorporated herein by reference. The liposomes are prepared in an aqueous buffer containing an ammonium salt, such as ammonium sulfate, ammonium phosphate, ammonium citrate, etc., typically 0.1 to 0.3 M ammonium salt, at a suitable pH, e.g., 5.5 to 7.5. The gradient can also be produced by including in the hydration medium sulfated polymers, such as dextran sulfate ammonium salt, heparin sulfate ammonium salt or sucralfate. After liposome formation and sizing, the external medium is exchanged for one lacking ammonium ions. In this approach, during the loading the amphipathic weak base is exchanged with the ammonium ion.

Yet, another approach is described in U.S. Pat. No. 5,939,096, incorporated herein by reference. In brief, the method employs a proton shuttle mechanism involving the salt of a weak acid, such as acetic acid, of which the protonated form translocates across the liposome membrane to generate a higher inside/lower outside pH gradient. An amphipathic weak acid compound is then added to the medium to the pre-formed liposomes. This amphipathic weak acid accumulates in liposomes in response to this gradient, and may be retained in the liposomes by cation (i.e. calcium ions)-promoted precipitation or low permeability across the liposome membrane, namely, the amphipathic weak acid is exchanges with the acetic acid.

According to the invention, the cytotoxic drug is preferably an amphipathic weak base compound. A preferred group of such cytotoxic drugs are any biologically active anthracycline-based amphipathic compounds.

Anthracyclines-based compounds share a common four-ringed 7,8,9,10-tetrahydrotetracene-5,12-quinone structure, and usually require glycosylation on specific sites for biological activity. As other aromatic polyketides, anthracyclines are typically synthesized by type two iterative polyketide synthase complex (PKS) from two-carbon units which are added to the growing carbon chain in consecutive acetyl-unit condensations. The carbon chain is then cyclisized to form the aromatic polyketide backbone, aglycone, which is further tailored via additional modification reactions before proceeding to the final glycosylation pathway. The precursor of most anthracycline-type aromatic polyketides is aklavinone, or less frequently nogalamycinone, which has the aklavinone C-9R ethyl replaced by C-9S methyl group.

Anthracyclines-based cytotoxic drugs are typically pro-apoptotoci drugs inducing their effect by acting as topoisomerase II inhibitors. Thus, in accordance with the invention, the term “biologically active anthracycline-based amphipathic compounds” denotes any anthracycline-based amphipathic compounds which exhibit a pro-apoptotic effect, specifically a topoisomerase II inhibitory activity.

Other cytotoxic drugs which are not topoisomerase II inhibitors may include, without being limited thereto, topoisomerate I inhibitors, such as topotecane. Other drugs may include mitoxantrone and vincaalkeloid (e.g. vinblastine and vincristine, vinorelbine) all being an amphipathic weak base and may be actively loaded onto liposomes by pH or ammonium ion gradient.

Knowing the detailed basis for structural diversity of these compounds, mathematical approach suggests that more than 10,000 theoretical anthracycline-analog structures could be possible. Some members of the family which have been shown to be clinically important in cancer treatment include daunorubicin, doxorubicin, idarubicin, epirubicin, pirarubicin, zorubicin, aclarubicin, and caminomycin and nemorubicin.

Doxorubicin which is the specific cytotoxic drug exemplified herein, has the chemical name 8S,10S)-10-[(3-amino-2,3,6-trideoxy-a-L-lyxo-hexopyranosyl)oxy]-8-glycolyl-7,8,9,10-tetrahydro-6,8,11-trihydroxy-1-methoxy-5,12-naphthacenedione hydrochloride and its analogs are also known in the art. Analogs include mitoxantrone, daunorubicin and N-acetyl daunorubicin, N-acetyladriamycin. Other doxorubicin analogs are described in U.S. Pat. Nos. 4,672,057; 4,345,068; 4,314,054; 4,229,355; 4,216,157; 4,199,571; 4,138,480, 5,304,687; US2001/036923 (WO01/49698) and WO04/082579, all being incorporated herein by reference.

The cytotoxic drug may be carried by a liposome separate from the lipid structure carrying the apoptosis affecting lipid, but it is preferably carried by the same lipid assembly incorporating in its lipid membrane the apoptosis affecting lipid.

The term “carried by” as used herein denotes any type of interaction between the cytotoxic drug and the assembly, including, without being limited thereto, encapsulation, adhesion, adsorption, entrapment (either within the inner or outer wall of a liposomal assembly or in an intraliposomal aqueous phase) or embedment in the lipid layer, however, encapsulating in the internal aqueous phase of the lipid assembly is the preferred manner of carrying the drug.

According to a preferred embodiment of the invention, the composition comprises a liposome, the liposome comprising a membrane constituted from HSPC, ^{2k}PEG-DSPE and C₆ ceramide and optionally a small amount of cholesterol (less than 5 mole % of the total lipid). According to a further preferred embodiment, the mole % of C₆ ceramide is about 11.5% of the total lipid.

The pharmaceutical composition may also comprise a physiologically acceptable carrier. Physiologically acceptable carriers generally refer to inert, non-toxic solid or liquid substances used to facilitate the delivery of the active entity (in this case the lipid assembly/liposomes included in the composition) to their target site. Those versed in the art of lipid-based drug delivery systems will know how to select the appropriate carriers in order to achieve the effective delivery of same.

As indicated above, the pro-apoptotic lipid and the cytotoxic drug may be carried by the same lipid assembly or by different populations of lipid assemblies (e.g. two types of liposomes). The lipid assemblies/liposomes carrying the apoptosis-affecting lipid and/or the cytotoxic drug are, at times, collectively termed herein “active entities”.

The amount of the active entities in the composition may be determined in appropriately designed clinical trials (dose range studies) and the person versed in the art will know how to properly conduct such trials in order to determine the effective amount. As generally known, an effective amount depends on a variety of factors including the distribution profile of the lipid structures within the body, a variety of pharmacological parameters such as half life in the body, undesired side effects, if any, on factors such as age and gender of the treated individual etc. The amount must be effective to achieve a desired therapeutic effect such as improved survival rate or more rapid recovery of the treated subject, or improvement or elimination of symptoms and other indicators associated with the condition under treatment, selected as appropriate measures by those skilled in the art.

Further, the pharmaceutical composition of the invention is administered and dosed taking into account the clinical condition of the individual, the site and method of administration, scheduling of administration, patient age, sex, body weight and other factors known to medical practitioners. The dosage form may be single dosage form or a multiple dosage form to be provided over a period of several days. The schedule of treatment with the pharmaceutical composition of the invention generally has a length proportional to the length of the disease process, the parameters of the individual to be treated (e.g. age and gender) and the effectiveness of the specific apoptosis-affecting lipid and cytotoxic drug employed.

The combination of the lipid assemblies carrying a pro-apoptotic lipid and a cytotoxic drug (either together or in separate lipid assemblies/liposomes) was shown to be effective in killing cancer cells as well as increasing the survival rate of tumor-bearing mice. Thus, the present invention also provides a method for treating a subject having proliferative or hyperproliferative conditions comprising administering to the subject the pharmaceutical composition of the invention.

Thus, the present invention preferably concerns the combination of a cytotoxic drug (as defined) with a pro-apoptotic lipid, the combination being preferably for the treatment of proliferative or hyper-proliferative conditions.

The term “proliferative or hyper-proliferation condition” or in short “hyper-proliferation condition” denotes any pathological condition manifested by the undesired cellular proliferation or hyperproliferation (accelerated growth and reproduction) or excessive accumulation of cells and which require for their treatment the inductions of apoptosis.

There are a variety of pathological conditions which are related to accelerated growth and reproduction of cells. For example, the hyperproliferative condition may be cancer. Any form of cancer is contemplated for treatment by the methods of the present invention. Cancers can be carcinomas, e.g., but not limited to, acinar carcinoma, adenocystic carcinoma, adenosquamous carcinoma, adnexal carcinoma, alveolar carcinoma, apocrine carcinoma, basal cell carcinoma, bladder carcinoma, breast carcinoma, bronchioloalveolar carcinoma, bronchogenic carcinoma, cervical carcinoma, colon carcinoma, cholangiocellular carcinoma, chorionic carcinoma, clear cell carcinoma, colloid carcinoma, cribriform carcinoma, ductal carcinoma, embryonal carcinoma, carcinoma en cuirasse, endometroid carcinoma, epidermoid carcinoma, esophageal carcinoma, carcinoma ex pleomorphic adenoma, follicular carcinoma of thyroid gland, gastric carcinoma, hepatocellular, carcinoma, carcinoma in situ, intraductal carcinoma, Hurthle cell carcinoma, inflammatory carcinoma of the breast, large cell carcinoma, lung carcinoma, invasive lobular carcinoma, lobular carcinoma, medullary carcinoma, meningeal carcinoma, Merkel cell carcinoma, mucinous carcinoma, mucoepidermoid carcinoma, nasopharyngeal carcinoma, non-small cell carcinoma, oat cell carcinoma, pancreatic carcinoma, papillary carcinoma, prostate carcinoma, renal cell carcinoma, scirrhous carcinoma, sebaceous carcinoma, carcinoma simplex, signet-ring cell carcinoma, small cell carcinoma, spindle cell carcinoma, squamous cell carcinoma, terminal duct carcinoma, transitional cell carcinoma, tubular carcinoma, and verrucous carcinoma.

Cancers can also be sarcomas, e.g., but not limited to, alveolar soft part sarcoma, ameloblastic sarcoma, botryoid sarcoma, clear cell sarcoma of kidney, endometrial stromal sarcoma, Ewing's sarcoma, giant cell sarcoma, hemangioendothelial sarcoma, immunoblastic sarcoma of B cells, immunoblastic sarcoma of T cells, Kaposi's sarcoma, Kupffer cell sarcoma, osteogenic sarcoma, pseudo-Kaposi sarcoma, reticulum cell sarcoma, Rous sarcoma, soft tissue sarcoma, and spindle cell sarcoma.

Other cancers that can be treated by the methods of the invention include, but are not limited to, retinoblastoma, neuroblastoma, and glioblastoma; leukemia and lymphoma.

The invention can also be applicable to treat hyperproliferative conditions that are not cancers, e.g., diseases or conditions involving stenosis. For example, the methods of the invention can be used to treat or prevent re-stenosis that occurs in a blood vessel, such as, but not limited to, that which occurs following balloon angioplasty or other treatments that cause injury to the blood vessels. Other examples of stenosis that can be treated in accordance with the present invention include, but are not limited to, aortic stenosis, hypertrophic pyloric stenosis, infantile hypertrophic gastric stenosis, mitral stenosis, pulmonary stenosis, pyloric stenosis, subaortic stenosis, renal artery stenosis, and tricuspid stenosis.

Yet, other conditions which may be treated in accordance with the invention are proliferative skin disorders, such as psoriasis, atopic dermatitis, allergic contact dermatitis, irritant contact dermatitis and further eczematous dermatitises, seborrhoeic dermatitis.

Yet, other conditions which may be treated in accordance with the invention are prolifertive ocular disorders such as diabetic retinopathy

The hyperproliferative condition is to be treated or prevented by the use of the composition of the invention.

Treatment or prevention in the context of the invention denotes any therapeutic effect achieve by the administration of the composition to a subject, which may be preventive, alleviating the disease or at least one of its undesired side effects, reducing the severity of the disease or the duration of its acute phase or cure altogether. This term includes: inhibition of growth, proliferation, and reproduction of cells associated with the pathological condition; induce programmed cell death at the diseased tissue or of the pathological cells, thereby eliminating or reducing the size of the pathological tissue etc, inhibition of the organization of the cells to undesired tissues or the neo-vascularization, and the change of the balance towards more differentiated cells. As may be appreciated by those versed in the art, the effect of the combined delivery of the active entities in accordance with the invention may be achieve any one of the following: to prevent manifestation of symptoms associated with the pathological condition before they occur; ameliorate undesired symptoms associated with the condition; slow down deterioration of such symptoms; slow down the progression of the condition; enhance onset of remission periods of a condition, slow down or prevent any irreversible damage caused by the condition, lessen the severity of the condition, improve survival rate and more rapid recovery from the condition or prevent the condition from occurring or any combination of the above.

Any conventional pharmaceutical practice may be employed to administer the present invention's compositions to subjects. Any appropriate route of administration may be employed, for example, but not limited to, intravenous, parenteral, transcutaneous, subcutaneous, intramuscular, intracranial, intraorbital, ophthalmic, intraventricular, intracapsular, intraspinal, intracisternal, intraperitoneal, intranasal, intrarectal, intravaginal, aerosol, or oral administration. A preferred mode of administration is injection, more preferably intravenous (i.v.) injection.

### DETAILED DESCRIPTION OF EXEMPLARY EMBODIMENTS

Materials and Methods

Materials: Hydrogenated soybean phosphatidylcholine (HSPC) was obtained from Lipoid KG (Ludwigshafen, Germany); N-carbamyl-poly-(ethylene glycol methyl ether)-1,2-distearoyl-sn-glycero-3-phosphoethanolamine triethyl ammonium salt (^{2K}PEG-DSPE) (the polyethylene glycol moiety having a molecular mass of 2000 Da) was obtained from Genzyme (Liestal, Switzerland); Cholesterol was purchased from Sigma; N-hexanoyl-D-erythro-sphingosine (C₆-Cer) was obtained from Biolab (Jerusalem, Israel).

Liposome preparation: The following liposomal formulations were prepared: HSPC:^{2k}PEG-DSPE:Chol:C₆Cer (76:7.5:5:11.5 mole % of the total components), HSPC:^{2k}PEG-DSPE:C₆Cer (81:7.5:11.5 mole %), HSPC:^{2k}PEG-DSPE:C₆Cer (78.5:10:11.5 mole %), DSPC:^{2k}PEG-DSPE:C₆Cer (81:7.5:11.5 mole %) and HSPC:^{2k}PEG-DSPE:Chol:C₆Cer (66:6.5:4.5:23 mole %).

Briefly, appropriate amounts of lipid stock solutions (appropriate for forming the above lipid ratios) were dissolved in ethanol mixed together in a test tube at appropriate molar ratios and heated at 60° C. The resulting solution was then added to aqueous ammonium sulphate buffer (250 mM, pH 5.0) by gentle mixing and heating at 60° C. for 1 hr to reach final ethanol concentration of 10% thereby obtaining multilamellar vesicles.

Large unilamellar vesicles (LUV ˜100 nm) were then prepared by extrusion of the MLV 10 times through 0.4-μm- and then 10 times through 0.1-μm-pore-size filters (Poretics, Livermore, Calif., USA) using for small scale of 1-2 ml the extrusion system of Avanti Polar Lipids (Alabaster, Ala.), or for larger volumes the Northern Lipids Inc. (Vancouver BC, Canada) extruder (scales 2-10 ml and 10-100 ml).

Remote loading of Doxorubicin (DXN): The remote loading procedure has been well characterized for amphipathic weak bases such as anthracyclines (Barenholz et al., U.S. Pat. No. 5,316,771, U.S. Pat. No. 5,192,549, incorporated herein by reference). Briefly, following hydration of lipids with ammonium sulphate (250 mM, pH 5.0) and extrusion, ammonium sulfate gradients were formed and ethanol was removed by dialysis 3 times against 200 volumes of 0.9% NaCl for 1 h each followed by a single, 24 h long, dialysis against 400 volumes of 10% sucrose. Then histidine buffer (pH 6.7) was added to the liposomes to a final concentration of 10 mM. The resulting liposomes exhibited a very large (>1000) trans-membrane ammonium sulfate gradient ([ammonium sulfate]ₗᵢₚₒₛₒₘₑₛ>>[ammonium sulfate]_{medium}) which induce a large (>3 pH units) proton gradient. An amount of 10 mM DXN solution was then added to the liposomes by incubation at 60° C. for 1 hr with gentle vortexing.

Liposome characterization: Liposomes were characterized for their particle size distribution (at 25° C.) by dynamic light-scattering (DLS) using the ALV-NIBS/BPPS ALV-Laser, Vertriebsgesellschaft GmbH, (Langen, Germany) instrument (according to manufacturer's instructions), and for their ceramide content using quantitative TLC. Specifically, ceramide was resolved using a solvent system composed of chloroform/methanol (95:5 v/v). The TLC plate was then sprayed with Copper sulfate reagent (composed of 100 g anhydrous copper sulfate containing 80 ml of phosphoric acid (85%), dissolved in 600 ml of highly pure (18.2 mega ohm) water), and the sprayed plates were heated and lipids appeared as dark brown spot. The spot absorbance was proportional to ceramide level which was compared to standard curve of appropriate ceramide. Silica gel plates 60 F₂₅₄ from Merk (Darsmstadt, Germany) were used and the ceramide spot absorbance (OD) was quantified using Fluor-S-MultiImiger (Bio-RAD, CA). The concentration of the total phospholipids (PL) which include PC and ^{2k}PEG-DSPE was verified by lipid phosphorus content determination which includes modified Bartlett method) [Shmeeda et al., 2003, Methods in Enzymol. 367, 272-292].

Determination of pH gradient. [Padan E, et al. J Biol Chem, 253 (1978): 3281-6] Ammonium sulfate and pH transmembrane gradients were determined. To this end, either ¹⁴C methylamine (MA) or acridine orange (AO) distribution between liposomes and medium was determined. This was determined in liposomes lacking or having ammonium sulfate gradient, and for the latter before and after DXN remote loading. In the case of ¹⁴C MA distribution studies, incubation was carried out for 30 min at 37° C. Then samples were passed (by centrifugation) down sephadex G-50 mini spin columns to separate liposome encapsulated ¹⁴[C]-methylamine from free unencapsulated ¹⁴C methylamine. The actual radioactivity in the liposomes was measured by β-counting (KONTRON Liquid Scintillation Counter). The pH gradient was calculated from the ratio of ¹⁴C methylamine/PL after and before separation on the Sephadex G50. A calibration curve in which both pHᵢₙ and pH_{med} are known was used in the liposomes lacking or containing DXN. The ¹⁴C MA distribution method was used to determine transmembrane pH gradient.

Accumulation of AO inside liposomes as a function of the ammonium sulfate gradient was studied in liposomes lacking DXN. The fluorescence emission intensity of acridine orange at 525 m was measured for the excitation wavelength of 490 nm in a 1-ml, quartz cuvette by LS50B luminescence spectrometer (Perkin Elmer, Norwalk, Conn.). First, the fluorescence intensity of the AO solution was recorded for 30 s at 60° C. (F⁰), then, liposomes were added and the fluorescence intensity (F) was monitored until it reached its equilibrium value. The data analysis assumed that fluorescence quenching is caused by the transfer of AO molecules from the external compartment into the liposome internal aqueous space and its aggregation due to the ammonium sulfate gradient [Clerc, S, and Barenholz, Y. (1998) Anal. Biochem. 259, 104-111]. The inside-to-outside mass ratio of AO was calculated from the following formula F/F⁰.

Determination of level of liposomes encapsulation and rate of release of DXN: The level of encapsulation and of the rate of release of DXN from liposomes containing DXN was measured using the cation exchanger Dowex 50X4-400 (Aldrich Chemical Company, Inc.), as described by Druckman et al. (1989) Biochim. Biophys. Acta 980:381-384; Amselem et al. (1990) J. Pharm. Sci. 79:1045-1052]. The ratio of 1 mg/50 mg between DXN and Dowex was used. Liposomes containing DXN were incubated with Dowex (50×4-400) for 10 min with gentle shaking and after that centrifugated at 5000 rpm for 2 min. DXN concentration in the liposomes was calculated from the absorbance measurements at 480 nm by Synergy HT plate reader in its absorbance mode (Bio-Tek, Winooski, Vt., USA) where the molar extinction of doxorucbin at 480 nm is 12500 O.D. % of free DXN in each liposome preparation was calculated in the liposomes by determining DXN level before and after the Dowex cation exchanger addition.

Size distribution analysis of LUV in the presence of serum: LUV of various defined compositions were incubated for up to 24 hr with adult calf serum (ACS) (Biological Industries Beit-HaEmek, Israel) at 25% and 50% (by volume) ACS, respectively. LUV-serum interactions were evaluated by monitoring changes in the liposome particle size at 25° C. using dynamic light scattering method by ALV-NIBS/HPPS ALV-Laser, Vertriebsgesellschaft GmbH, (Langen, Germany).

Determination of release of DXN from liposomes in the presence of serum: Various liposomes (see Table 1 for liposome composition) were incubated in the presence of serum for 2, 4, 24 and 72 hr at 37° C. After indicated time periods samples were interacted with DOWEX 50 cation exchanger (which binds free DXN but not liposomal DXN) for 10 min with gentle shaking followed by centrifugation at 5000 rpm for 2 min. Thereafter, samples were diluted 10-fold in 90% isopropanol containing 10% 0.75N HCl (ISP-HCl) in order to dissolve all liposome-lipids and release the DXN to the solution. Concentration of DXN in liposomes was compared to concentration of DXN in liposomes which were mixed with the cation exchanger Dowex 50 and determined according to a standard calibration curve of DXN (exitation at 485±10 nm and emission at 590±10 nm) from fluorescence intensity using Synergy HT plate reader in its fluorescence mode (Bio-Tek, Vermont, USA).

Cytotoxicity studies: The cytotoxicity of C₆Cer-DXN-SSL against doxorubicin-resistant human breast carcinoma M-109 cell line was tested by the methylene blue (MB) staining assay [Gorodetsky, R. et al. Int. J. Cancer. 75:635-642 (1998)]. A known number of exponentially growing cells in 200 μL of medium were plated in 96-microwell, flat-bottomed plates. For each of the variants tested, 4 wells were used. Following 24 hr of incubation in culture, different concentrations of drugs or formulations were added to each well containing untreated cells.

Cells were exposed to drugs for 96 hr. At the end of drug exposure the drug-treated cells, as well as parallel control cells, were washed, and the incubation continued in fresh medium until termination of the experiment. Following 96 hr of growth, cells were fixed by adding 50 μL of 2.5% glutaraldehyde to each well for 15 min. Fixed cells were rinsed 10 times with deionized water and once with borate buffer (0.1 M, pH 8.5), dried, and stained with MB (100 μL of 1% solution in 0.1 M borate buffer, pH 8.5) for 1 h at room temperature (r.t.). Stained cells were rinsed thoroughly with de-ionized water to remove any non-cell-bound dye and then dried. The MB bound to the fixed cells was extracted by incubation at 37° C. with 200 μL of 0.1 N HCl for 1 h, and the net optical density of the dye in each well was determined by a plate spectrophotometer (Labsystems Multyskan Bichromatic, Finland) at 620 nm.

In vivo evaluation of antitumor efficacy of Liposomal DXN: All the experimental procedures which make use of animals (mice) were done in accordance with the standards required by the Institutional Animal Care and Use Committee of the Hebrew University and Hadassah Medical Organization and approved by the Committee.

To test therapeutic efficacy, female BALB/c mice (in the weight range of 16-20 g) were injected i.p. with 1*10⁶ C-26 colon carcinomas. The viability of these cells was >90% by trypan blue exclusion. Therapeutic efficacy of i.v. injected SSL (DXN-C₆Cer-SSL) containing both DXN in intraliposome aqueous phase (DXN/PL ratio of 0.2) and 11.5 mole % of C₆Cer in comparison to Doxil (alone) and free DXN was studied. In all 3 treatments DXN dose was 0.16 mg/mouse (8 mg/kg) and dose of C₆Cer injected into mice treated with DXN-C₆Cer-SSL was 0.25 mg.mouse (12.5 mg/kg). Intravenous treatment began at day 4 after tumor cell inoculation and was repeated three times at 5-days intervals. The median survival and percentage increase in life span of treated (T) over control (C) animals (Tx100/C)-100 were calculated.

Pharmacokinetics and biodistribution studies in tumor-bearing mice: Eight to 10-week-old BALB/c female mice, obtained through the Animal Breeding House of the Hebrew University (Jerusalem, Israel), were housed at Hadassah Medical Center at a specific pathogen free (SPF) facility with food and water ad libitum. Each mouse was injected with one inoculum of tumor cells (1×10⁶ murine C-26 cells) subcutaneously into the left flank. 7 days later SSL formulations containing both DXN (0.16 mg/mouse) and C₆Cer, (0.25 mg/mouse), Doxil (0.16 mg/mouse) and free DXN (0.16 mg/mouse) were injected i.v. At 1, 4, 24 and 48 hr after injection, the animals were anesthetized with 4% chloral hydrate (Fluka, USA), bled by eye inoculation, and plasma was separated from blood cells by 5 min centrifugation at 5,000 rpm. Various organs (liver, heart, lungs, kidneys) and tumor were removed. Each time period in each treated group consisted of 3 mice. Samples were frozen at −80° C. until assayed. Thereafter, plasma samples were diluted in 90% isopropanol containing 10% 0.75N HCl in order to dissolve all liposome's lipids and release the DXN to the solution, and concentration of DXN was determined according to a standard calibration curve of DXN (excitation at 485±10 mu and emission at 590±10 nm) from fluorescence intensity using Synergy HT plate reader in its fluorescence mode (Bio-Tek, Vermont, USA).

Statistical analysis: Median survival times and the statistical significance of differences in survival curves were calculated by means of the log-rank test using Prism Software (GraphPad, San Diego, Calif.). Differences were considered significant at P<0.05. For assessment of synergy, the combination index (CI) was determined by median effect analysis [Chow T C, Talalay P. Quantitative analysis of dose-effect relationships: the combined effects of multiple drugs or enzyme inhibitors. Adv Enzyme Regul 22:27-55 (1984)]. The equation used to calculate the combination index was CI=(D₁/Dx₁)+(D₂/Dx₂)+(D₁D₂/Dx₁Dx₂), where Dx is the individual drug concentration at its respective IC₅₀ and D is the concentration of drug in the combination that results in 50% growth inhibition.

Results

Cytotoxicity studies: An initial in vitro study was performed to examine the combined effect of liposomal C₆Cer and Doxorubicin. It was found that IC50 and IC10 values of liposomal C₆Cer are 3.1 and 1.4 μM, respectively. Dose response curves were then generated with DXN given alone (1 μM, 2.5 μM or 5 μM) or in combination with liposomal C₆Cer at its IC₁₀. The in vitro results presented in FIG. 1 show that administration of liposomal C₆Cer and DXN have an additive effect on survival of breast cancer M-109 doxorubicin-resistant cell line. This is evident from the IC₅₀ value for free DXN being 2.2 μM when given in free from and alone, vs. an IC50 value of 0.9 μM in the presence of IC₁₀ concentration of liposomal C₆Cer (FIG. 1).

Combinatory index (CI) [Modralk D E, et al. Synergistic interaction between sphingomyelin and gemcitabine potentiates ceramide-mediated apoptosis in pancreatic cancer. Cancer Res. 2004 Nov. 15; 64(22):8405-10] was determined to be equal to 1.0. Considering that a CI value <0.9 indicates synergism, a CI value between 0.9 and 1.1 indicates additivity, and a CI value >1.1 indicates antagonism, the resulting CI indicates an additive interaction between liposomal C₆Cer and DXN in this cell line.

Characterization of liposomal formulations: SSL (100 nm) composed of HSPC or of DSPC liposome-forming lipids, stabilized by ^{2k}PEG-DSPE and having either 11.5 or 23 mole % of C₆Cer were successfully formed. For further study, HSPC as the liposome-forming lipid, the lipopolymer ^{2k}PEG-DSPE (7.5 mole %) and C₆Cer, were used. In some formulations, a low amount of cholesterol (5 mole %) in the SSL was used. As a cytotoxic drug, DXN was used.

As described above, DXN was introduced into preformed SSL by an active (remote) loading using liposome high/medium low transmembrane ammonium ion gradient. To measure the transmembrane pH the distribution of ¹⁴[C]-methylamine that was added to liposomes having ammonium sulfate gradient before and after DXN loading was determined. Approximately 80% of the ¹⁴[C]-methylamine was distributed into the liposomes lacking DXN while post-DXN loading only about 30% of ¹⁴[C]-methylamine distributed into the liposomes. Based on the calibration curve, this suggests a pH gradient of 3.3 pH units before loading, and of only 1.4 pH units post loading under conditions that the medium pH in both cases was 6.7.

These results indicate that all the liposomal formulation tested and described herein had a high (90-95%) encapsulation of DXN (assessed by cation exchanger Dowex 50 that binds all free DXN) and C₆Cer (assessed by TLC).

LUV stability: Stability of liposomal formulations during storage at 4° C. was evaluated by measuring particle size distribution using dynamic light scattering and by determining percent of free DXN, which was assayed by addition of the cation exchanger Dowex 50 to the SSL formulation. It was found that SSL formulations containing 11.5 mole % of C₆Cer were physically stable for 8 months. While SSL formulations containing 23 mole % of C₆Cer were unstable, as release of its C₆Cer occurs already after 3 weeks of storage at 4° C., although these liposome formulation preserve its transmembrane pH gradient (determined by ¹⁴[C]-methylamine distribution (see Materials and Methods)) for at least 3 months, which suggests that the release of part of the C₆Cer did not disturb the barrier properties of the liposomes. Liposomal formulations consisting of HSPC:^{2k}PEG-DSPE:C₆Cer (81:7.5:11.5 mole %), HSPC:^{2k}PEG-DSPE:C₆Cer (78.5:10:11.5 mole %) and of DSPC:^{2k}PEG-DSPE:C₆Cer (81:7.5:11.5 mole %) were stable for at least 3 month (still ongoing experiment).

Measurement of size distribution of SSL formulations in serum: As the SSL formulations are aimed for intravenous (i.v.) administration it was important to study and evaluate the effect of serum on the physical stability of the C₆Cer-DXN-SSL in comparison to Doxil. Therefore, changes in size of different 100 mu SSL formulations varying in their composition as result of their exposure to adult calf serum (ACS) was measured by dynamic light scattering as described in Materials and Methods. It was found, and as also detailed in Table 1 below, that the size of all SSL formulations did not change significantly when brought into contact with serum able below).

TABLE 1
effect of serum on size distribution of SSL formulations:

| | | | Size |
|---|---|---|---|
| | Initial | Size in | in |
| | size | 25% | 50% |
| LUV Formulation (mole %) | (nm) | ACS | ACS |
| HSPC:^{2k}PEG-DSPE:Chol:C₆Cer (76:7.5:5:11.5)- | 100 | 96 | 100 |
| DXN | | | |
| HSPC:^{2k}PEG-DSPE:C₆Cer (81:7.5:11.5)-DXN | 88 | 94 | 94 |
| HSPC:^{2k}PEG-DSPE:C₆Cer (78.5:10:11.5)-DXN | 100 | 88 | 94 |
| DSPC:^{2k}PEG-DSPE:C₆Cer (81:7.5:11.5)-DXN | 92 | 84 | 96 |
| Doxil (HSPC:Chol:^{2k}PEG-DSPE (54.5:40:5.5) | 84 | 96 | 84 |
| DXN | | | |

Release of DXN from liposomes in the presence of serum: The basic requirement of liposomal utilization is that they have to retain the drug inside the liposome, which, on the one hand allows to bring the maximum drug to the target site, and on the other hand, to reduce drug toxicity and therefore increase therapeutic index of the drug. Therefore an aim was to determine the rate of DXN release from various liposomes prepared, in comparison to release of the drug from Doxil formulation. The results show that rate of DXN release from DXN-SSL having 11.5 mole % of C₆Cer as well as from Doxil was very low. Further, it was found that after 72 hr incubation of the various SSL with serum, 95-97% of DXN retained in the liposomes, independent on the composition of lipid in the liposomal formulation.

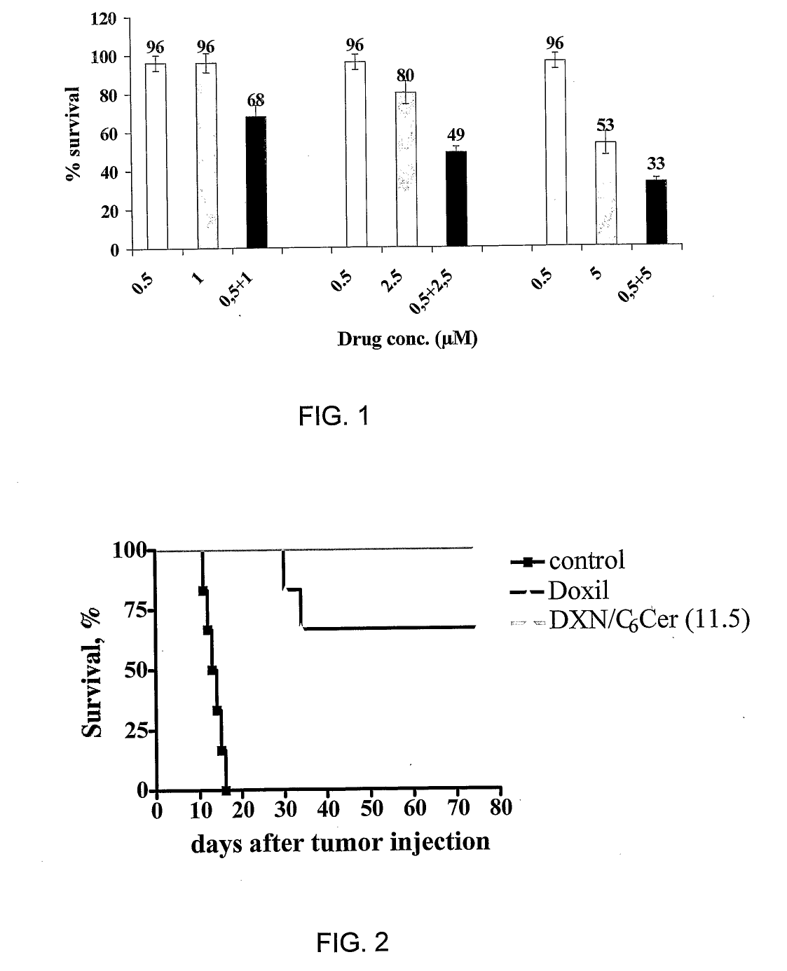

Therapeutic efficacy evaluation of the liposomes containing DXN and C₆Cer in the same SSL in comparison to Doxil in mice tumor model: To test therapeutic efficacy, female BALB/c mice were injected i.p. with 1*10⁶ C-26 murine colon carcinoma. Therapeutic efficacy of i.v. injected SSL containing both DXN and C₆Cer (in the membrane) in comparison to Doxil was then determined. The results, presented in FIG. 2, demonstrate that 100% (p***<0.0005) of mice treated with SSL containing both DXN and 11.5 mole % of C₆Cer (HSPC:^{2k}PEG-DSPE:Chol:C₆Cer (76:7.5:5:11.5)-DXN) survived for 2 month (long-term survival) as compared to 70% (p***<0.0005) survival in the case of mice treated with Doxil (liposomal DXN).

SSL containing 23 mole % of C₆Cer encapsulating DXN showed less effective anticancer activity, with only 17% long-term survival observed in comparison to Doxil or to SSL containing 11.5 mole % of C₆Cer encapsulating DXN.

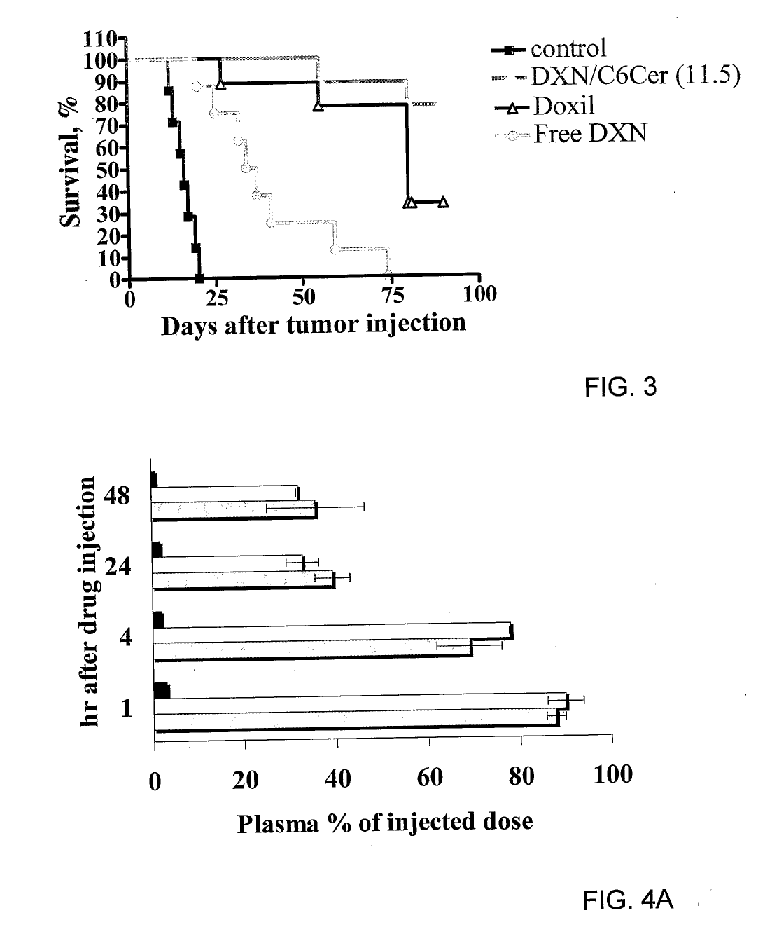

Long term survival of tumor-bearing mice (over 60 day after tumor injection) was also examined. Comparison of efficacy between Doxil and SSL-C₆Cer-DXN demonstrated that 80 days after treatment initialization, 75% of mice treated with both types of SSL formulations survived (p***<0.0001), compared to no (0%) survival with untreated mice or with mice treated with free DXN. Ninety days post treatment only 25% of mice treated with Doxil survived compared to 75% those treated with SSL-C₆Cer-DXN. These results are demonstrated in FIG. 3.

When comparing median survival times of treated and untreated groups, 80 day median survival was found in group treated with Doxil compared to 35-day and 16 day survival of groups treated with free DXN and untreated ones, respectively. In mice treated with liposomal formulation containing both DXN and C₆Cer the median survival was longer than 80 day and therefore, undefined.

Doxorubicin pharmacokinetics and biodistribution studies in tumor-bearing mice: BALB/c female mice were injected with one inoculum of tumor cells (1×10⁶ C-26 cells) subcutaneously into the left flank. Seven days later, liposomal formulations containing both DXN and C₆Cer [HSPC:^{2k}PEG-DSPE:Chol:C₆Cer (76:7.5:5:11.5)-DXN]; Doxil or Free DXN were injected i.v., and bled by eye inoculation, plasma was isolated and 50 μl was taken from plasma for further analysis. DXN levels were determined after “extraction” of plasma with acidic isopropanol as described in Methods.

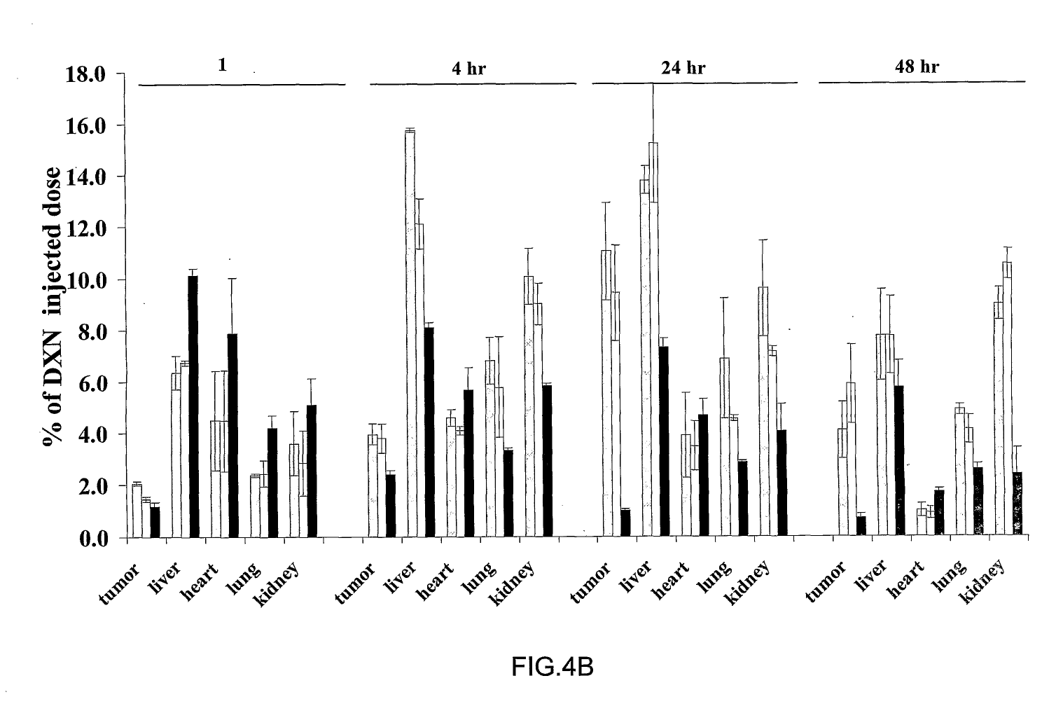

Pharmacokinetics studies revealed long circulation time of DXN delivered via SSL-C₆Cer-DXN which was comparable to Doxil and much longer than for free DXN. It was found that 48 hr post-injection 36% and 32% of DXN delivered by SSL-C₆Cer-DXN or Doxil, respectively, remained in circulation, as compared with 3% obtained with free DXN remaining in plasma 1 hr post-injection (plasma levels of free DXN at 48 hours were below detection). The results are presented in FIG. 4A.

The biodistribution of DXN delivered to various organs and tumor tissue by both types of SSL formulations (HSPC:^{2k}PEG-DSPE:Chol:C₆Cer (76:7.5:5:11.5)-DXN, or Doxil) and by free DXN was determined at different time points and the results are presented in FIG. 4B. As shown, free DXN was cleared much faster by kidneys then SSL-C₆Cer-DXN, and much higher levels of free DXN delivered as free drug were detected in heart tissue (compare 5.7% of free DXN and 1.6 and 1.1% of injected SSL-C₆Cer-DXN or Doxil, respectively, at 4 hr post-treatment). This suggests reduced cardiac toxicity of the SSL-C₆Cer-DXN and Doxil, compared with free DXN. On the other hand, due to much longer circulation time of both types of SSL significantly higher levels of DXN were found in tumor tissue at all time periods tested reaching maximum at 24 hr post-injection (compare 11% and 9.4% in case of injected SSL-C₆Cer-DXN or Doxil, respectively, to 1% in the case of injected free DXN).

Therefore, it was concluded that sterically stabilized (SSL)-Ceramide comprising liposomes encapsulating DXN or Doxil accumulate in tumor at much higher level than free DXN and, this explain superior therapeutic activity, and, also, reduced systemic and cardiac toxicity of DXN delivered via SSL compared with free DXN.

1-75. (canceled)

76. A pharmaceutical composition comprising a short chain ceramide selected from C₂, C₄, C₆ or C₈-ceramide, and a lipopolymer forming part of a liposome's membrane, the liposome encapsulating a cytotoxic, amphipathic weak base drug.

77. The pharmaceutical composition of claim 76, wherein said short chain ceramide has a hydrophobic region and a polar headgroup, the atomic mass ratio between the headgroup and hydrophobic region being less than 0.3.

78. The pharmaceutical composition of claim 76, wherein said cytotoxic drug is an anthracycline-based drug.

79. The pharmaceutical composition of claim 78, wherein the cytotoxic, amphipathic weak base drug is doxorubicin.

80. The pharmaceutical composition of claim 76, wherein said lipopolymer has a hydrophobic lipid region and a polymer headgroup, wherein the atomic mass ratio between the headgroup and hydrophobic region is at least 1.5.

81. The pharmaceutical composition of claim 76, wherein said lipopolymer has a level of water, tightly bound to its headgroup, of at least about 0.60 molecules of water per lipopolymer headgroup.

82. The pharmaceutical composition of claim 81, wherein said polymer headgroup is polyethylene glycol (PEG).

83. The pharmaceutical composition of claim 82, wherein said PEG has an atomic mass of 2,000 Da (^{2k}PEG).

84. The pharmaceutical composition of claim 76, wherein said liposome membrane comprises a phospholipid.

85. The pharmaceutical composition of claim 84, wherein said phospholipid is a glycerophospholipid.

86. The pharmaceutical composition of claim 85, wherein said glycerophospholipid is hydrogenated soybean phosphatidylcholine (HSPC).

87. The pharmaceutical composition of claim 76, wherein said ceramide is C₆ ceramide.

88. The pharmaceutical composition of claim 87, wherein said C₆ ceramide is present in said membrane at a molar % of 11.5% of total lipid.

89. The pharmaceutical composition of claim 88, wherein said liposome is stable for at least 6 months when incubated with serum or plasma at 37° C. with respect to size.

90. The pharmaceutical composition of claim 76, wherein said liposome comprises cholesterol at a mole % which is equal or less than 5%.

91. A pharmaceutical composition comprising a short N-acyl chain ceramide or a pro-apoptotic lipid selected from ceramines, sphinganines, sphinganine-1-phosphate, di- or tri-alkylshpingosines and their structural analogs, and a lipopolymer forming part of the liposome's membrane, the liposome encapsulating cytotoxic, amphipathic weak base drug.

92. The pharmaceutical composition of claim 90, wherein the cytotoxic, amphipathic weak base drug is doxorubicin.

93. The pharmaceutical composition of claim 90, wherein said lipopolymer has a hydrophobic lipid region and a polymer headgroup, wherein the atomic mass ratio between the headgroup and hydrophobic region is at least 1.5.

94. The pharmaceutical composition of claim 93, wherein said polymer headgroup is polyethylene glycol (PEG).

95. The pharmaceutical composition of claim 94, wherein said PEG has an atomic mass of 2,000 Da (^{2k}PEG).

96. The pharmaceutical composition of claim 90, wherein said liposome membrane comprises a phospholipid.

97. The pharmaceutical composition of claim 96, wherein said phospholipid is a glycerophospholipid.

98. The pharmaceutical composition of claim 97, wherein said glycerophospholipid is hydrogenated soybean phosphatidylcholine (HSPC).

99. The pharmaceutical composition of claim 90, wherein said ceramide is C₆ ceramide.

100. The pharmaceutical composition of claim 99, wherein said C₆ ceramide is present in said membrane at a molar % of 11.5% of total lipid.

101. The pharmaceutical composition of claim 90, wherein said liposome comprises cholesterol at a mole % which is equal or less than 5%.

102. A method for the treatment of a disease or disorder comprising administering to a subject in need of said treatment a composition according to claim 76.

103. The method of claim 102, wherein said ceramide is C₆ ceramide, said lipopolymer is PEG and said cytotoxic amphipathic weak base drug is doxorubicin.

104. A method for the treatment of a disease or disorder comprising administering to a subject in need of said treatment a composition according to claim 90.

105. The method of claim 104, wherein said ceramide is C₆ ceramide, said lipopolymer is PEG and said cytotoxic amphipathic weak base drug is doxorubicin.
